(19) United States
(12) Patent Application Publication
(10) Pub. No.: US 2014/0100422 A1
(43) Pub. Date: Apr. 10, 2014

(54) IN-VIVO INFORMATION ACQUISITION SYSTEM AND METHOD FOR CONTROLLING IN-VIVO INFORMATION ACQUISITION SYSTEM

(71) Applicant: OLYMPUS CORPORATION, Tokyo (JP)

(72) Inventors: Hideharu MIYAHARA, Kamiina-gun (JP); Kenji MIYATA, Okaya-shi (JP)

(73) Assignee: OLYMPUS CORPORATION, Tokyo (JP)

(21) Appl. No.: 14/107,078

(22) Filed: Dec. 16, 2013

Related U.S. Application Data
(63) Continuation of application No. PCT/JP2012/063714, filed on May 29, 2012.

(30) Foreign Application Priority Data
Jun. 16, 2011 (JP) 2011-134396

(51) Int. Cl. A61B 1/00

(52) U.S. Cl. CPC A61B 1/00016
USPC 600/101

(57) ABSTRACT

An in-vivo information acquisition system is an in-vivo information acquisition system including an in-vivo information acquisition apparatus provided with a configuration capable of transmitting a wireless signal which includes in-vivo information about an inside of a subject, and a terminal apparatus provided with a configuration capable of receiving the wireless signal transmitted from the in-vivo information acquisition apparatus outside the subject, wherein the terminal apparatus includes a reception confirmation signal transmission section generating and transmitting a reception confirmation signal when the wireless signal which includes the in-vivo information about the subject can be received; and the in-vivo information acquisition apparatus includes a storage section, and a control section causing the wireless signal which includes the in-vivo information about the subject to be transmitted and at the same time, causing the same in-vivo information as included in the transmitted wireless signal to be stored into the storage section at least during a period during which the reception confirmation signal cannot be received.

### CROSS REFERENCE TO RELATED APPLICATION

This application is a continuation application of PCT/JP2012/063714 filed on May 29, 2012 and claims benefit of Japanese Application No. 2011-134396 filed in Japan on Jun. 16, 2011, the entire contents of which are incorporated herein by this reference.

### BACKGROUND OF THE INVENTION

1. Field of the Invention

The present invention relates to an in-vivo information acquisition system, and more particularly, to an in-vivo information acquisition system capable of acquiring in-vivo information and a method for controlling the in-vivo information acquisition system.

2. Description of the Related Art

Endoscopes in a medical field have been conventionally used for the purpose of in-vivo observation and the like. As one kind of the above endoscopes, a capsule endoscope has been proposed in recent years which is arranged in a body cavity by being swallowed by a subject, can pick up images of an object while moving through the body cavity accompanying vermiculation, and wirelessly transmit the picked-up images of the object to an outside as image-pickup signals.

As a capsule endoscope having a configuration substantially similar to the capsule endoscope described above, for example, the capsule endoscope disclosed in Japanese Patent Application Laid-Open Publication No. 2005-342083 is given.

More specifically, Japanese Patent Application Laid-Open Publication No. 2005-342083 discloses a configuration of a system including a capsule-type medical apparatus acquiring in-vivo information of a subject and an in-vivo information receiving apparatus receiving the in-vivo information, wherein a communication confirmation signal for confirming condition of communication with the in-vivo information receiving apparatus is transmitted from the capsule-type medical apparatus; a communication permission signal for permitting communication is transmitted from the in-vivo information receiving apparatus when the communication confirmation signal is received; and the in-vivo information is transmitted from the capsule-type medical apparatus when the communication permission signal is received.

### SUMMARY OF THE INVENTION

An in-vivo information acquisition system of an aspect of the present invention is an in-vivo information acquisition system including an in-vivo information acquisition apparatus provided with a configuration capable of transmitting a wireless signal which includes in-vivo information about an inside of a subject acquired by an in-vivo information acquisition section to an outside of the subject, and a terminal apparatus provided with a configuration capable of receiving the wireless signal transmitted from the in-vivo information acquisition apparatus outside the subject, wherein the terminal apparatus includes a reception confirmation signal transmission section generating a reception confirmation signal and transmitting the reception confirmation signal to the in-vivo information acquisition apparatus when the wireless signal which includes the in-vivo information about the subject can be received; and the in-vivo information acquisition apparatus includes a storage section, and a control section causing the wireless signal which includes the in-vivo information about the subject to be transmitted to the outside of the subject and at the same time, causing the same in-vivo information as included in the wireless signal transmitted to the outside of the subject to be stored into the storage section at least during a period during which the reception confirmation signal transmitted from the terminal apparatus cannot be received.

An in-vivo information acquisition system control method of an aspect of the present invention is a method for controlling an in-vivo information acquisition system including an in-vivo information acquisition apparatus provided with a configuration capable of transmitting a wireless signal which includes in-vivo information about an inside of a subject acquired by an in-vivo information acquisition section to an outside of the subject, and a terminal apparatus provided with a configuration capable of receiving the wireless signal transmitted from the in-vivo information acquisition apparatus outside the subject, the method including the steps of: a reception confirmation signal being generated and transmitted to the in-vivo information acquisition apparatus by an operation of a reception confirmation signal transmission section of the terminal apparatus when the wireless signal which includes the in-vivo information about the subject is received; the wireless signal which includes the in-vivo information about the subject being transmitted to the outside of the subject by an operation of a control section of the in-vivo information acquisition apparatus at least during a period during which the reception confirmation signal transmitted from the terminal apparatus cannot be received; and the same in-vivo information as included in the wireless signal transmitted to the outside of the subject being stored into a storage section by an operation of the control section at least during the period during which the reception confirmation signal transmitted from the terminal apparatus cannot be received.

### BRIEF DESCRIPTION OF THE DRAWINGS

FIG. 1 is a diagram showing a configuration of main parts of an in-vivo information acquisition system in a first embodiment of the present invention;

FIG. 2 is a schematic diagram for illustrating an aspect of use of the in-vivo information acquisition system;

FIG. 3 is a timing chart for illustrating operations of an in-vivo information acquisition apparatus and a terminal apparatus in the first embodiment; and

FIG. 4 is a timing chart for illustrating operations of an in-vivo information acquisition apparatus and a terminal apparatus in a second embodiment.

### DETAILED DESCRIPTION OF THE PREFERRED EMBODIMENTS

Embodiments of the present invention will be described below with reference to drawings.

### First Embodiment

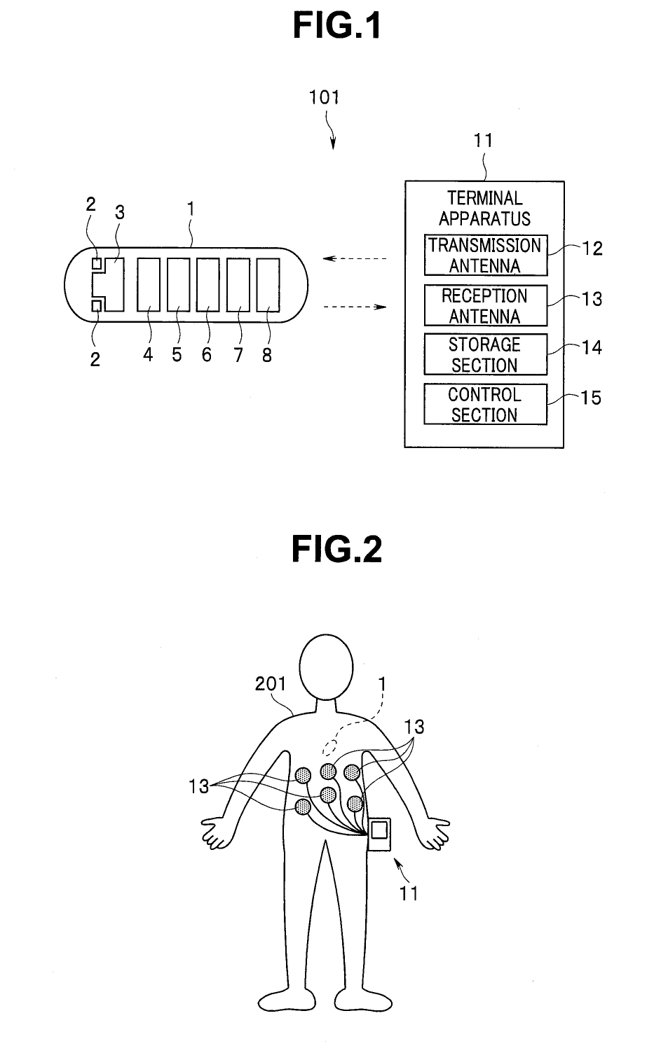

FIGS. 1 to 3 relate to a first embodiment of the present invention.

FIG. 1 is a diagram showing a configuration of main parts of an in-vivo information acquisition system in the first embodiment of the present invention.

An in-vivo information acquisition system 101 is configured being provided with an in-vivo information acquisition apparatus 1 and a terminal apparatus 11 as shown in FIG. 1.

The in-vivo information acquisition apparatus 1 is configured as an apparatus having a size, shape and the like which can be arranged in a body cavity of a subject, for example, like a capsule endoscope.

The in-vivo information acquisition apparatus 1 is configured being provided with an illumination section 2 emitting illuminating light for illuminating an object in the body cavity of the subject, an image pickup section 3 picking up the object illuminated by the illumination section 2 and acquiring image data, a wireless transmission section 4 modulating the image data acquired by the image pickup section 3 to a wireless signal and transmitting the wireless signal to the terminal apparatus 11, a wireless reception section 5 receiving the wireless signal transmitted from the terminal apparatus 11 and demodulating the wireless signal, a storage section 6 capable of accumulating image data acquired by the image pickup section 3, a power source section 7 capable of supplying drive power required for driving each section of the in-vivo information acquisition apparatus 1, and a control section 8 performing control related to operations of each section of the in-vivo information acquisition apparatus 1.

That is, an in-vivo information acquisition section in the present embodiment is configured being provided with the illumination section 2 and the image pickup section 3.

The terminal apparatus 11 is configured as a portable apparatus which can be driven by power supplied from an internal battery not shown, for example, like a mobile terminal apparatus.

The terminal apparatus 11 is configured being provided with a transmission antenna 12 modulating a reception conformation signal to be described later to a wireless signal and transmitting the wireless signal to the in-vivo information acquisition apparatus 1, a reception antenna 13 receiving the wireless signal transmitted from the in-vivo information acquisition apparatus 1 and demodulating the wireless signal, a storage section 14 capable of accumulating image data received by the reception antenna 13, and a control section 15 performing control related to operations of each section of the terminal apparatus 11 including generation of a reception confirmation signal to be described later.

That is, a reception confirmation signal transmission section in the present embodiment is configured being provided with the transmission antenna 12 and the control section 15.

Note that the in-vivo information acquisition apparatus 1 in the present embodiment is not limited to one configured so as to operate in response to drive power supplied from the power source section 7, which is an internal battery or the like. For example, the in-vivo information acquisition apparatus 1 may be configured so as to operate in response to drive power supplied from the outside by wireless power supply utilizing an electromagnetic phenomenon.

FIG. 2 is a schematic diagram for illustrating an aspect of use of the in-vivo information acquisition system.

The in-vivo information acquisition system 101 of the present embodiment is used for a subject 201, for example, according to an aspect shown in FIG. 2. More specifically, at the time of using the in-vivo information acquisition system 101, the in-vivo information acquisition apparatus 1 is arranged in the body cavity of the subject 201; a body part of the terminal apparatus 11 is fixed to the outside of the body of the subject 201; and a plurality of the reception antennas 13 connected via signal lines extending from the body part of the terminal apparatus 11, respectively, are arranged on a body surface part of the subject 201.

Note that the body part of the terminal apparatus 11 is not limited to one configured so as to be usable in a state of being fixed to the subject 201. The body part of the terminal apparatus 11 may be configured as such that is on the assumption of being used in a state of being set at a predetermined position, for example, like a workstation.

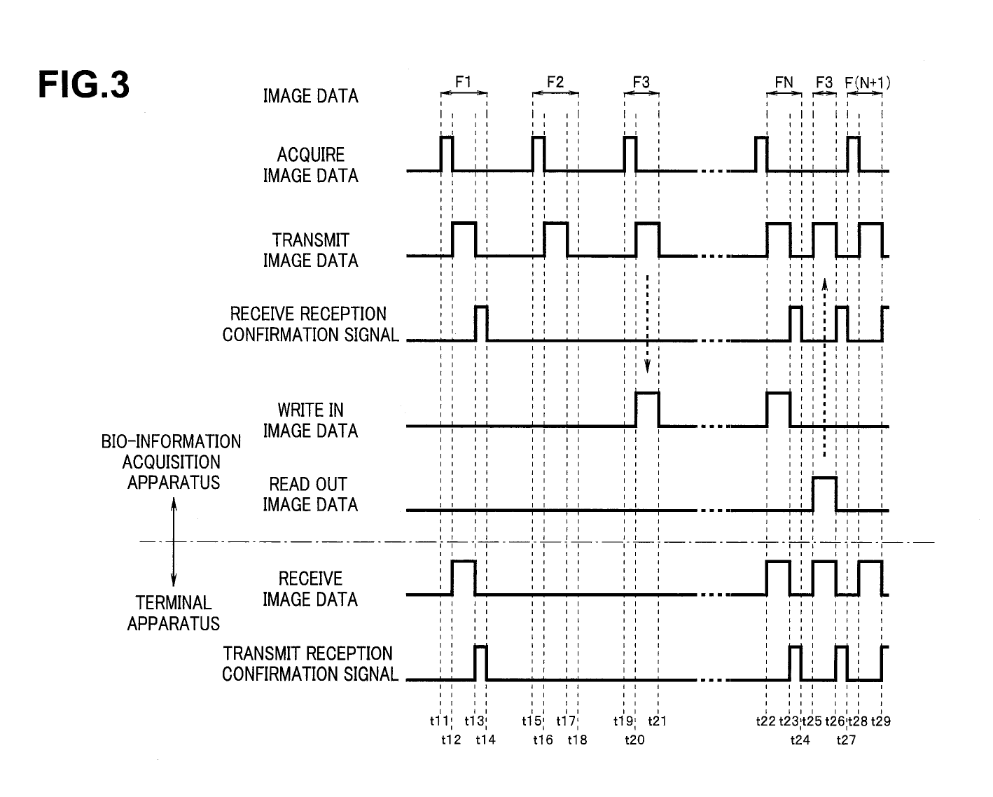

Here, specific operations of the in-vivo information acquisition apparatus 1 and the terminal apparatus 11 in the present embodiment will be described with appropriate reference to a timing chart in FIG. 3. FIG. 3 is a timing chart for illustrating operations of the in-vivo information acquisition apparatus and the terminal apparatus in the first embodiment.

First, at arbitrary timing before time t11, each of power sources for the in-vivo information acquisition apparatus 1 and the terminal apparatus 11 is on, and each of the in-vivo information acquisition apparatus 1 and the terminal apparatus 11 is arranged so that the in-vivo information acquisition apparatus 1 and the terminal apparatus 11 have a positional relationship illustrated in FIG. 2.

After that, the control section 8 of the in-vivo information acquisition apparatus 1 performs control for causing the image pickup section 3 to pick up an image of an object in the body cavity of the subject 201 to acquire image data F1 during a period from the time t11 to time t12. Furthermore, the control section 8 of the in-vivo information acquisition apparatus 1 performs control for causing the image data F1 acquired by the image pickup section 3 to be transmitted from the wireless transmission section 4 to the terminal apparatus 11 during a period from the time t12 to time t13.

If a wireless signal which includes the image data F1 can be received by the reception antennas 13 during the period from the time t12 to the time t13, the control section 15 of the terminal apparatus 11 causes the image data F1 to be stored into the storage section 14. Furthermore, the control section 15 of the terminal apparatus 11 performs control for causing a reception confirmation signal indicating that the wireless signal which includes the image data F1 could be received to be generated and transmitted from the transmission antenna 12 to the in-vivo information acquisition apparatus 1 during a period from the time t13 to time t14. Then, immediately after transmission of the reception confirmation signal indicating that the wireless signal which includes the image data F1 could be received is completed, the control section 15 of the terminal apparatus 11 transitions to a state of waiting to receive next image data.

If the reception confirmation signal can be received by the wireless reception section 5 during the period from the time t13 to the time t14, the control section 8 of the in-vivo information acquisition apparatus 1 obtains a judgment result that the condition of communication between the in-vivo information acquisition apparatus 1 and the terminal apparatus 11 is good, that is, the image data F1 acquired by the image pickup section 3 has been stored into the terminal apparatus 11.

Next, the control section 8 of the in-vivo information acquisition apparatus 1 performs control for causing the image pickup section 3 to pick up an image of the object in the body cavity of the subject 201 to acquire image data F2 during a period from time t15 to time t16. Furthermore, the control section 8 of the in-vivo information acquisition apparatus 1 performs control for causing the image data F2 acquired by the image pickup section 3 to be transmitted from the wireless transmission section 4 to the terminal apparatus 11 during a period from the time t16 to time t17, on the basis of the judgment result that the image data F1 has been stored into the terminal apparatus 11.

On the other hand, if a wireless signal which includes the image data F2 cannot be received by the reception antenna 13 after the time t14, the control section 15 of the terminal apparatus 11 maintains the state of waiting to receive next image data without performing generation and transmission of a reception confirmation signal.

If a reception confirmation signal cannot be received by the wireless reception section 5 during a period after completion of transmission of the image data F2 until time t18 before start of acquisition of next image data F3, the control section 8 of the in-vivo information acquisition apparatus 1 obtains a judgment result that the condition of communication between the in-vivo information acquisition apparatus 1 and the terminal apparatus 11 is bad, that is, the image data F2 acquired by the image pickup section 3 has not been stored into the terminal apparatus 11.

Next, the control section 8 of the in-vivo information acquisition apparatus 1 performs control for causing the image pickup section 3 to pick up an image of the object in the body cavity of the subject 201 to acquire the image data F3 during a period from time t19 to time t20. Furthermore, the control section 8 of the in-vivo information acquisition apparatus 1 performs control for causing the image data F3 acquired by the image pickup section 3 to be transmitted from the wireless transmission section 4 to the terminal apparatus 11 and at the same time, causing the image data F3 to be stored into the storage section 6 during a period from the time t20 to time t21, on the basis of the judgment result that the image data F2 has not been stored into the terminal apparatus 11.

After that, as the control section 8 of the in-vivo information acquisition apparatus 1 continues to receive a judgment result that image data has not been stored into the terminal apparatus 11 because a reception confirmation signal cannot be received by the wireless reception section 5, the control section 8 of the in-vivo information acquisition apparatus 1 performs control so as to cause image data F4, F5, . . . , F(N−1) temporally sequentially acquired by the image pickup section 3 to be transmitted from the wireless transmission section 4 to the terminal apparatus 11 as well as causing the image data F4, F5, . . . , F(N−1) to be stored into the storage section 6. That is, if the condition of communication between the in-vivo information acquisition apparatus 1 and the terminal apparatus 11 is bad, the control section 8 of the in-vivo information acquisition apparatus 1 operates so as to cause image data acquired by the image pickup section 3 to be stored into the storage section 6.

Then, the control section 8 of the in-vivo information acquisition apparatus 1 performs control for causing image data FN acquired by the image pickup section 3 to be transmitted from the wireless transmission section 4 to the terminal apparatus 11 and at the same time, causing the image data FN to be stored into the storage section 6 during a period from time t22 to time t23, on the basis of a judgment result that the image data F(N−1) has not been stored into the terminal apparatus 11.

If a wireless signal which includes the image data FN can be received by the reception antenna 13 during the period from the time t22 to the time t23, the control section 15 of the terminal apparatus 11 causes the image data FN to be stored into the storage section 14. Furthermore, the control section 15 of the terminal apparatus 11 performs control for causing a reception confirmation signal indicating that the wireless signal which includes the image data FN could be received to be generated and transmitted from the transmission antenna 12 to the in-vivo information acquisition apparatus 1 during a period from the time t23 to time t24. Then immediately after transmission of the reception confirmation signal indicating that the wireless signal which includes the image data FN could be received is completed, the control section 15 of the terminal apparatus 11 transitions to the state of waiting to receive next image data.

If the reception confirmation signal can be received by the wireless reception section 5 during the period from the time t23 to the time t24, the control section 8 of the in-vivo information acquisition apparatus 1 obtains a judgment result that condition of communication between the in-vivo information acquisition apparatus 1 and the terminal apparatus 11 is good, that is, the image data FN acquired by the image pickup section 3 has been stored into the terminal apparatus 11.

Then, the control section 8 of the in-vivo information acquisition apparatus 1 performs control for causing the image data F3 stored in the storage section 6 to be read out and transmitted from the wireless transmission section 4 to the terminal apparatus 11 during a period from time t25 to time t26, which is a period included in anywhere between a timing of receiving the reception confirmation signal for the image data FN and a timing of starting acquisition of next image data F(N+1), on the basis of the judgment result that the image data FN has been stored into the terminal apparatus 11.

If a wireless signal which includes the image data F3 can be received by the reception antenna 13 during the period from the time t25 to the time t26, the control section 15 of the terminal apparatus 11 causes the image data F3 to be stored into the storage section 14. Furthermore, the control section 15 of the terminal apparatus 11 performs control for causing a reception confirmation signal indicating that the wireless signal which includes the image data F3 could be received to be generated and transmitted from the transmission antenna 12 to the in-vivo information acquisition apparatus 1 during a period from the time t26 to time t27. Then, immediately after transmission of the reception confirmation signal indicating that the wireless signal which includes the image data F3 could be received is completed, the control section 15 of the terminal apparatus 11 transitions to the state of waiting to receive next image data.

The control section 8 of the in-vivo information acquisition apparatus 1 obtains a judgment result that the image data F3 read from the storage section 6 has been stored into the terminal apparatus 11, on the basis of the fact that the reception confirmation signal could be received by the wireless reception section 5 during the period from the time t26 to the time t27. Then, the control section 8 of the in-vivo information acquisition apparatus 1 performs control for causing the image pickup section 3 to pick up an image of the object in the body cavity of the subject 201 to acquire the image data F(N+1) during a period from time t27 to time t28 immediately after acquisition of the judgment result that the image data F3 has been stored into the terminal apparatus 11. Furthermore, the control section 8 of the in-vivo information acquisition apparatus 1 performs control for causing the image data F(N+1) acquired by the image pickup section 3 to be transmitted from the wireless transmission section 4 to the terminal apparatus 11 during a period from the time t28 to time t29.

After that, as the control section 8 of the in-vivo information acquisition apparatus 1 continues to receive a judgment result that image data has been stored into the terminal apparatus 11 because a reception confirmation signal could be received by the wireless reception section 5, the control section 8 of the in-vivo information acquisition apparatus 1 performs control for causing the image data F4, F5, . . . , F(N−1) stored in the storage section 6 to be sequentially read out as well as causing the image data F4, F5, . . . , F(N−1) read out from the storage section 6 and image data newly acquired by the image pickup section 3 to be alternately transmitted from the wireless transmission section 4 to the terminal apparatus 11. That is, when the condition of communication between the in-vivo information acquisition apparatus 1 and the terminal apparatus 11 is improved and becomes good, the control section 8 of the in-vivo information acquisition apparatus 1 operates so as to cause groups of image data stored into the storage section 6 during a period during which the condition of communication between the in-vivo information acquisition apparatus 1 and the terminal apparatus 11 is bad to be sequentially read out and transmitted from the wireless transmission section 4 to the terminal apparatus 11.

Note that the control section 15 of the terminal apparatus 11 of the present embodiment is not limited to one that operates so as to cause one reception confirmation signal to be generated and transmitted when a wireless signal which includes image data can be received once. For example, the control section 15 may operate so as to cause one reception confirmation signal to be generated and transmitted when a wireless signal which includes image data can be received a predetermined number of times equal to twice or more.

Furthermore, the control section 8 of the in-vivo information acquisition apparatus 1 of the present embodiment is not limited to one that performs control related to transmission of image data each time the image pickup section 3 acquires image data once during a period during which a reception confirmation signal cannot be received. For example, the control section 8 may perform the control related to transmission of image data each time the image pickup section 3 acquires image data a predetermined number of times equal to twice or more.

Furthermore, the control section 8 of the in-vivo information acquisition apparatus 1 of the present embodiment may delete image data accumulated in the storage section 6 all together when it can be confirmed that all image data acquired by the image pickup section 3 has been stored into the terminal apparatus 11. Alternatively, the control section 8 of the in-vivo information acquisition apparatus 1 of the present embodiment may divide a group of image data accumulated in the storage section 6 to sequentially delete the divided image data multiple times when it can be confirmed that image data acquired by the image pickup section 3 has been stored into the terminal apparatus 11.

As described above, the in-vivo information acquisition system 101 of the present embodiment has a configuration capable of using a wireless signal which includes image data obtained by actually picking up an image of an object to confirm whether the condition of communication between the in-vivo information acquisition apparatus 1 and the terminal apparatus 11 is good or bad. Therefore, according to the in-vivo information acquisition system 101 of the present embodiment, it is possible to simplify control related to communication operations between the in-vivo information acquisition apparatus 1 and the terminal apparatus 11 in comparison with conventional systems as well as reducing power consumed at the time of the communication operations between the in-vivo information acquisition apparatus 1 and the terminal apparatus 11 in comparison with the conventional systems.

### Second Embodiment

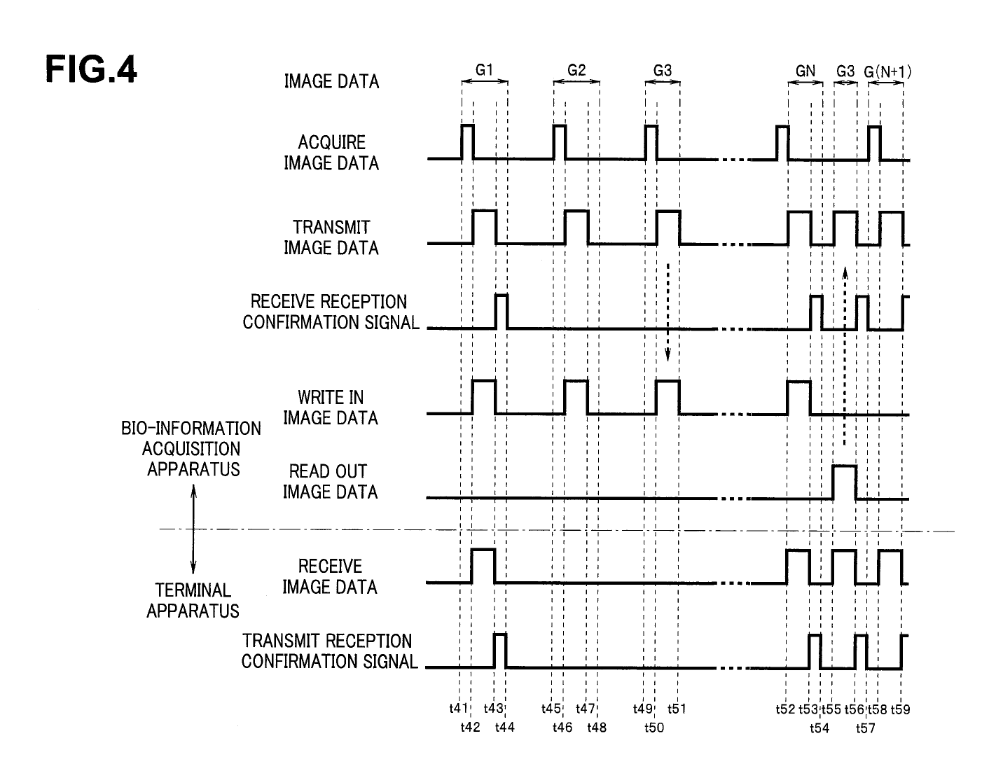

FIG. 4 relates to a second embodiment of the present invention. Note that an in-vivo information acquisition system in the present embodiment has a configuration substantially similar to that of the in-vivo information acquisition system 101 in the first embodiment. Therefore, in the present embodiment, description will be made mainly on parts which perform operations different from those of the in-vivo information acquisition system 101 in the first embodiment.

Here, specific operations of an in-vivo information acquisition apparatus 1 and a terminal apparatus 11 in the present embodiment will be described with appropriate reference to a timing chart in FIG. 4. FIG. 4 is a timing chart for illustrating operations of the in-vivo information acquisition apparatus and the terminal apparatus in the second embodiment.

First, at arbitrary timing before time t41, each of power sources for the in-vivo information acquisition apparatus 1 and the terminal apparatus 11 is on, and each of the in-vivo information acquisition apparatus 1 and the terminal apparatus 11 is arranged so that the in-vivo information acquisition apparatus 1 and the terminal apparatus 11 have the positional relationship illustrated in FIG. 2.

After that, a control section 8 of the in-vivo information acquisition apparatus 1 performs control for causing an image pickup section 3 to pick up an image of an object in a body cavity of a subject 201 to acquire image data G1 during a period from the time t41 to time t42. Furthermore, the control section 8 of the in-vivo information acquisition apparatus 1 performs control for causing the image data G1 acquired by the image pickup section 3 to be transmitted from a wireless transmission section 4 to the terminal apparatus 11, and at the same time, performs control for causing the image data G1 to be stored into a storage section 6 during a period from the time t42 to time t43.

If a wireless signal which includes the image data G1 can be received by a reception antenna 13 during the period from the time t42 to the time t43, a control section 15 of the terminal apparatus 11 causes the image data G1 to be stored into a storage section 14. Furthermore, the control section 15 of the terminal apparatus 11 performs control for causing a reception confirmation signal indicating that the wireless signal which includes the image data G1 could be received to be generated and transmitted from a transmission antenna 12 to the in-vivo information acquisition apparatus 1 during a period from the time t43 to time t44. Then, immediately after transmission of the reception confirmation signal indicating that the wireless signal which includes the image data G1 could be received is completed, the control section 15 of the terminal apparatus 11 transitions to the state of waiting to receive next image data.

If the reception confirmation signal can be received by a wireless reception section 5 during the period from the time t43 to the time t44, the control section 8 of the in-vivo information acquisition apparatus 1 obtains a judgment result that condition of communication between the in-vivo information acquisition apparatus 1 and the terminal apparatus 11 is good, that is, the image data G1 acquired by the image pickup section 3 has been stored into the terminal apparatus 11 as well as giving identification information by which the judgment result can be identified, to the image data G1 stored in the storage section 6.

Next, the control section 8 of the in-vivo information acquisition apparatus 1 performs control for causing the image pickup section 3 to pick up an image of the object in the body cavity of the subject 201 to acquire image data G2 during a period from time t45 to time t46. Furthermore, the control section 8 of the in-vivo information acquisition apparatus 1 performs control for causing the image data G2 acquired by the image pickup section 3 to be transmitted from the wireless transmission section 4 to the terminal apparatus 11, and at the same time, performs control for causing the image data G2 to be stored into the storage section 6 during a period from the time t46 to time t47.

On the other hand, if a wireless signal which includes the image data G2 cannot be received by the reception antenna 13 after the time t44, the control section 15 of the terminal apparatus 11 maintains the state of waiting to receive next image data without performing generation and transmission of a reception confirmation signal.

If a reception confirmation signal cannot be received by the wireless reception section 5 during a period after completion of transmission of the image data G2 until time t48 before start of acquisition of next image data G3, the control section 8 of the in-vivo information acquisition apparatus 1 obtains a judgment result that the condition of communication between the in-vivo information acquisition apparatus 1 and the terminal apparatus 11 is bad, that is, the image data G2 acquired by the image pickup section 3 has not been stored into the terminal apparatus 11 as well as giving identification information by which the judgment result can be identified, to the image data G2 stored in the storage section 6.

Next, the control section 8 of the in-vivo information acquisition apparatus 1 performs control for causing the image pickup section 3 to pick up an image of the object in the body cavity of the subject 201 to acquire image data G3 during a period from time t49 to time t50. Furthermore, the control section 8 of the in-vivo information acquisition apparatus 1 performs control for causing the image data G3 acquired by the image pickup section 3 to be transmitted from the wireless transmission section 4 to the terminal apparatus 11 and at the same time, causing the image data G3 to be stored into the storage section 6 during a period from the time t50 to time t51.

After that, as the control section 8 of the in-vivo information acquisition apparatus 1 continues to receive a judgment result that image data has not been stored into the terminal apparatus 11 because a reception confirmation signal cannot be received by the wireless reception section 5, the control section 8 of the in-vivo information acquisition apparatus 1 causes image data G4, G5, . . . , G(N−1) temporally sequentially acquired by the image pickup section 3 to be transmitted from the wireless transmission section 4 to the terminal apparatus 11 as well as giving identification information by which the judgment result can be identified, to the image data G3, G4, G5, . . . , G(N−1) stored in the storage section 6.

Then, the control section 8 of the in-vivo information acquisition apparatus 1 performs control for causing the image data GN acquired by the image pickup section 3 to be transmitted from the wireless transmission section 4 to the terminal apparatus 11 and at the same time, causing the image data GN to be stored into the storage section 6 during a period from time t52 to time t53.

If a wireless signal which includes the image data GN can be received by the reception antenna 13 during the period from the time t52 to the time t53, the control section 15 of the terminal apparatus 11 causes the image data GN to be stored into the storage section 14. Furthermore, the control section 15 of the terminal apparatus 11 performs control for causing a reception confirmation signal indicating that the wireless signal which includes the image data GN could be received to be generated and transmitted from the transmission antenna 12 to the in-vivo information acquisition apparatus 1 during a period from the time t53 to time t54. Then immediately after transmission of the reception confirmation signal indicating that the wireless signal which includes the image data GN could be received is completed, the control section 15 of the terminal apparatus 11 transitions to the state of waiting to receive next image data.

If the reception confirmation signal can be received by the wireless reception section 5 during the period from the time t53 to the time t54, the control section 8 of the in-vivo information acquisition apparatus 1 obtains a judgment result that the condition of communication between the in-vivo information acquisition apparatus 1 and the terminal apparatus 11 is good, that is, the image data GN acquired by the image pickup section 3 has been stored into the terminal apparatus 11 as well as giving identification information by which the judgment result can be identified, to the image data GN stored in the storage section 6.

Then, the control section 8 of the in-vivo information acquisition apparatus 1 performs control for causing the image data G3 to which information of not being stored in the terminal apparatus 11 is given to be read out from the storage section 6 and transmitted from the wireless transmission section 4 to the terminal apparatus 11 during a period from time t55 to time t56, which is a period included in anywhere between a timing of receiving the reception confirmation signal for the image data GN and a timing of starting acquisition of next image data G(N+1), on the basis of the judgment result that the image data GN has been stored into the terminal apparatus 11.

If a wireless signal which includes the image data G3 can be received by the reception antenna 13 during the period from the time t55 to the time t56, the control section 15 of the terminal apparatus 11 causes the image data G3 to be stored into the storage section 14. Furthermore, the control section 15 of the terminal apparatus 11 performs control for causing a reception confirmation signal indicating that the wireless signal which includes the image data G3 could be received to be generated and transmitted from the transmission antenna 12 to the in-vivo information acquisition apparatus 1 during a period from the time t56 to time t57. Then immediately after transmission of the reception confirmation signal indicating that the wireless signal which includes the image data G3 could be received is completed, the control section 15 of the terminal apparatus 11 transitions to the state of waiting to receive next image data.

The control section 8 of the in-vivo information acquisition apparatus 1 obtains a judgment result that the image data G3 has been stored into the terminal apparatus 11 as well as updating the identification information given to the image data G3 in the storage section 6, on the basis of the fact that the reception confirmation signal could be received by the wireless reception section 5 during the period from the time t56 to the time t57. Then, the control section 8 of the in-vivo information acquisition apparatus 1 performs control for causing the image pickup section 3 to pick up an image of the object in the body cavity of the subject 201 to acquire the image data G(N+1) during a period from time t57 to time t58 immediately after acquisition of the judgment result that the image data G3 has been stored into the terminal apparatus 11. Furthermore, the control section 8 of the in-vivo information acquisition apparatus 1 performs control for causing the image data G(N+1) acquired by the image pickup section 3 to be transmitted from the wireless transmission section 4 to the terminal apparatus 11 during a period from the time t58 to time t59.

After that, as the control section 8 of the in-vivo information acquisition apparatus 1 continues to receive a judgment result that image data has been stored into the terminal apparatus 11 because a reception confirmation signal could be received by the wireless reception section 5, the control section 8 of the in-vivo information acquisition apparatus 1 performs control for causing the image data G4, G5, . . . , G(N−1) to which the information of not being stored in the terminal apparatus 11 is given to be sequentially read out from the storage section 6 as well as causing the image data G4, G5, . . . , G(N−1) read out from the storage section 6 and image data newly acquired by the image pickup section 3 to be alternately transmitted from the wireless transmission section 4 to the terminal apparatus 11. That is, when the condition of communication between the in-vivo information acquisition apparatus 1 and the terminal apparatus 11 is improved and becomes good, the control section 8 of the in-vivo information acquisition apparatus 1 operates so as to cause groups of image data stored into the storage section 6 during a period during which the condition of communication between the in-vivo information acquisition apparatus 1 and the terminal apparatus 11 is bad to be sequentially read out and transmitted from the wireless transmission section 4 to the terminal apparatus 11.

Note that the control section 8 of the in-vivo information acquisition apparatus 1 of the present embodiment may delete a group of image data to which information of having been stored into the terminal apparatus 11 is given all together from the storage section 6 when it can be confirmed that all image data acquired by the image pickup section 3 has been stored into the terminal apparatus 11. Alternatively, the control section 8 of the in-vivo information acquisition apparatus 1 of the present embodiment may divide the group of image data to which the information of having been stored into the terminal apparatus 11 is given and sequentially delete the divided image data from the storage section 6 multiple times.

As described above, the in-vivo information acquisition system 101 of the present embodiment has a configuration capable of using a wireless signal which includes image data obtained by actually picking up an image of an object to confirm whether the condition of communication between the in-vivo information acquisition apparatus 1 and the terminal apparatus 11 is good or bad. Therefore, according to the in-vivo information acquisition system 101 of the present embodiment, it is possible to simplify control related to communication operations between the in-vivo information acquisition apparatus 1 and the terminal apparatus 11 in comparison with conventional systems as well as reducing power consumed at the time of the communication operations between the in-vivo information acquisition apparatus 1 and the terminal apparatus 11 in comparison with the conventional systems.

Furthermore, the in-vivo information acquisition system 101 of the present embodiment has a configuration capable of performing transmission of image data acquired by the image pickup section 3 to the terminal apparatus 11 and storage of the image data into the storage section 6 simultaneously or substantially simultaneously. Therefore, according to the in-vivo information acquisition system 101 of the present embodiment, it is possible to prevent missing of image data acquired by the image pickup section 3, for example, even under such an environment that the condition of communication between the in-vivo information acquisition apparatus 1 and the terminal apparatus 11 deteriorates intermittently.

Note that the present invention is not limited to each embodiment described above, and it goes without saying that various alterations and applications are possible within a range not departing from the spirit of the invention.

What is claimed is:

1. An in-vivo information acquisition system comprising an in-vivo information acquisition apparatus provided with a configuration capable of transmitting a wireless signal which includes in-vivo information about an inside of a subject acquired by an in-vivo information acquisition section to an outside of the subject, and a terminal apparatus provided with a configuration capable of receiving the wireless signal transmitted from the in-vivo information acquisition apparatus outside the subject, wherein
the terminal apparatus comprises a reception confirmation signal transmission section generating a reception confirmation signal and transmitting the reception confirmation signal to the in-vivo information acquisition apparatus when the wireless signal which includes the in-vivo information about the subject can be received; and
the in-vivo information acquisition apparatus comprises a storage section, and a control section causing the wireless signal which includes the in-vivo information about the subject to be transmitted to the outside of the subject and at the same time, causing the same in-vivo information as included in the wireless signal transmitted to the outside of the subject to be stored into the storage section at least during a period during which the reception confirmation signal transmitted from the terminal apparatus cannot be received.

2. The in-vivo information acquisition system according to claim 1, wherein the control section causes groups of in-vivo information stored in the storage section to be sequentially read out and wirelessly transmitted to the terminal apparatus after detecting transition from the period during which the reception confirmation signal transmitted from the terminal apparatus cannot be received to a period during which the reception confirmation signal can be received.

3. The in-vivo information acquisition system according to claim 1, wherein the control section causes the wireless signals that include the in-vivo information acquired by the in-vivo information acquisition section to be sequentially transmitted to the outside of the subject, causes the same in-vivo information as included in the wireless signals transmitted to the outside of the subject to be sequentially stored into the storage section, and further gives identification information capable of identifying whether in-vivo information is in-vivo information acquired during the period during which the reception confirmation signal transmitted from the terminal apparatus cannot be received, to each in-vivo information stored in the storage section.

4. The in-vivo information acquisition system according to claim 3, wherein the control section causes groups of in-vivo information acquired during the period during which the reception confirmation signal transmitted from the terminal apparatus cannot be received to be sequentially read out from the storage section and wirelessly transmitted to the terminal apparatus on the basis of the identification information after detecting transition from the period during which the reception confirmation signal transmitted from the terminal apparatus cannot be received to a period during which the reception confirmation signal can be received.

5. A method for controlling an in-vivo information acquisition system comprising an in-vivo information acquisition apparatus provided with a configuration capable of transmitting a wireless signal which includes in-vivo information about an inside of a subject acquired by an in-vivo information acquisition section to an outside of the subject, and a terminal apparatus provided with a configuration capable of receiving the wireless signal transmitted from the in-vivo information acquisition apparatus outside the subject, the method comprising the steps of:
a reception confirmation signal being generated and transmitted to the in-vivo information acquisition apparatus by an operation of a reception confirmation signal transmission section of the terminal apparatus when the wireless signal which includes the in-vivo information about the subject is received;
the wireless signal which includes the in-vivo information about the subject being transmitted to the outside of the subject by an operation of a control section of the in-vivo information acquisition apparatus at least during a period during which the reception confirmation signal transmitted from the terminal apparatus cannot be received; and
the same in-vivo information as included in the wireless signal transmitted to the outside of the subject being stored into a storage section by an operation of the control section at least during the period during which the reception confirmation signal transmitted from the terminal apparatus cannot be received.

6. The in-vivo information acquisition system control method according to claim 5, further comprising a step of groups of in-vivo information stored in the storage section being sequentially read out and wirelessly transmitted to the terminal apparatus by an operation of the control section after transition from the period during which the reception confirmation signal transmitted from the terminal apparatus cannot be received to a period during which the reception confirmation signal can be received is detected.

7. The in-vivo information acquisition system control method according to claim 5, further comprising the steps of:
the wireless signals that include the in-vivo information acquired by the in-vivo information acquisition section being sequentially transmitted to the outside of the subject by an operation of the control section;
the same in-vivo information as included in the wireless signals transmitted to the outside of the subject being sequentially stored into the storage section by an operation of the control section; and
identification information capable of identifying whether in-vivo information is in-vivo information acquired during the period during which the reception confirmation signal transmitted from the terminal apparatus cannot be received being given to each in-vivo information stored in the storage section by an operation of the control section.

8. The in-vivo information acquisition system control method according to claim 7, further comprising a step of groups of in-vivo information acquired during the period during which the reception confirmation signal transmitted from the terminal apparatus cannot be received being sequentially read out from the storage section and wirelessly transmitted to the terminal apparatus by an operation of the control section based on the identification information after transition from the period during which the reception confirmation signal transmitted from the terminal apparatus cannot be received to a period during which the reception confirmation signal can be received is detected.
